(19) United States
(12) Patent Application Publication
(10) Pub. No.: US 2007/0010842 A1
(43) Pub. Date: Jan. 11, 2007

(54) Safety trocar obturator

(76) Inventor: Sergey Popov, Beer Sheva (IL)

Correspondence Address: DANIEL J SWIRSKY, 55 REUVEN ST., BEIT SHEMESH, 99544, IL

(21) Appl. No.: 10/561,956

(22) PCT Filed: Jun. 21, 2004
(86) PCT No.: PCT/IL04/00554

§ 371 Date: May 15, 2006

(30) Foreign Application Priority Data
Jun. 22, 2003 (IL) 156582

(51) Int. Cl. A61B 17/34

(52) U.S. Cl. 606/185

(57) ABSTRACT

A trocar obturator for minimal invasive surgery (see FIG. 34) comprising: an apex knife adapted to carrying out an initial incision in a body cavity wall and a knife protecting shield movable between retracted and extended-protecting positions during patient's tissue penetrating. There is a means for locking the shield in its protecting position after piercing a patient's skin. In version embodiment the lock means includes a control knob for shield unlocking, fully disposed in the penetrating head at distal obturator part. This lock means design allows its using along with the knife and shield as the constituents of detachable penetrating subassembly adapted for mounting on the obturator by a quickly acting connector. After piercing the patient's skin, the shield is transformed into a blunt penetrating tip forming a passageway in patient's soft tissue and in peritoneum by a safe manner.

### BACKGROUND

1. Field of the Invention

The present invention relates to a safety trocar device for providing access into patient's body cavity during a minimal invasive surgical procedure.

2. Prior Art of the Invention

The general trend of the modern trocar development Is the use of the safety means preventing the patient's internal organs from injury by a trocar sharp tip. As a rule safety means substantially complicates the trocar design. This in full measure relates to the trocar versions with detachable distal components.

U.S. Pat. No. 5,697,947 discloses Trocar Obturator Including a Removable Knife, wherein the removable knife is connected to an obturator shaft by a connector including a bulbous knife protrusion, fitting and nut. The trocar is provided with a safety means in the form of a protective sleeve shielding the knife, extending through a trocar cannula and biased distally by two springs disposed at the trocar proximal portion. After use, the trocar assembly should be disassembled, cleaned, sterilized and reused. The disadvantage of the trocar is complexity of its design including about 17 separate details only in the obturator and safety means subassemblies. This substantially hampers the performance of the trocar disassembly, cleaning and subsequent assembly, needed for its reuse. Another disadvantage is relatively high cost of the trocar. Another disadvantage is the danger of the introducing and jamming the tissue fibers in the gaps between the knife and protective sleeve during penetrating the patient's body wall resulting in disturbing the normal operation of the knife and safety means. Another disadvantage is the large resistance to protective sleeve advance through a patent's body cavity wall because of its large diameter resulting in the delay of the knife protection and lowering the safety means effectiveness.

U.S. Pat. No. 5,709,671 discloses Trocar Having an Improved Tip Configuration, wherein there is a flat knife with a linear cutting edge including a penetrating apex and lateral beveled cutting edges. The knife is surrounded with a movable safety shield, which also fulfils a function of a dilator enlarging the opening in a body cavity wall to adapt it for subsequent cannula insertion. The shield biasing means is located beyond the obturator distal tip. The disadvantage of the trocar is the complexity of its obturator including two mutually movable shafts independently connecting the knife and the shield with the obturator handle. Another disadvantage is the impossibility of replacing the obturator distal tip without the replacement of the all obturator including its shafts and handle. These both disadvantages predetermine the increased cost of the trocar. Another disadvantage is the large resistance to safety shield advance into the body wall opening, resulting in the delay of the knife protection and lowering the safety means effectiveness. This large resistance arises as a result of using the safety shield as a dilator, as well as because of the shield disposal on all sides of the knife. Another disadvantage is the danger of the introducing and jamming the tissue fibers in the gap between the knife and safety shield during penetrating the patients body wall resulting in disturbing the normal operation of the knife and safety means. This gap is formed on the knife lateral side opposite to the disposition of its sharpened cutting edge.

### SUMMARY OF THE INVENTION

The objective of the present invention is enhancing the effectiveness of the safety means operation due to reducing the resistance to the shield advance into the body tissue.

Another objective is preventing the patient's body tissue from introducing and jamming between the elements of the obturator distal end, specifically between the penetrating apex and safety means, and thereby heightening the reliability of the trocar operation.

Another objective is simplifying the processes of the reusable trocar obturator disassembly, cleaning, sterilizing and subsequent assembly.

Another objective is improving the safety of the reusable trocar obturator disassembly and assembly.

Another objective is improving the manufacturing properties of the trocar obturator, including the minimization of its detail number, simplification of the detail design and obturator assembling, and the unification of the safety means, eventually resulting in the reduction of the manufacturing cost.

Another objective is improving the safety of the disposable detachable penetrating trocar units due to the full arrangement of the locking means in the distal trocar part.

Another objective is additional enhancing of the safety means operation due to locking the apex knife shield during its stay in the patient's soft tissue and subsequent using the apex knife shield as a penetrating blunt tip of the trocar obturator.

Another objective is extending the capabilities of the trocar obturator application at the expense of using the detachable penetrating head.

The above noted objectives are accomplished with a trocar obturator including: a shaft, an obturator handle disposed on the proximal end of the shaft, and a penetrating head disposed on the distal end of the shaft. The penetrating head comprises: a dilating sloping surface and consisting of a penetrating head base inseparably connected to the shaft and a penetrating head cover quickly detachable from the penetrating head base; an apex knife adapted to carrying out an incision in a body cavity wall and made as an apex knife body in the form of a metal plate with a sharpened cutting edge at its distal end detachably housed in the penetrating head, therewith the dilating sloping surface is immovable relative to the shaft during the trocar operation; a safety means for protecting at least the apex knife. The safety means is completely disposed at the penetrating head, has at least an apex knife shield designed for the protection of the apex knife, and comprises a biasing spring. The apex knife shield has the width substantially equal to the width of the apex knife and less than maximal diameter of the penetrating head, and moreover, the apex knife shield width exceeds its thickness. The apex knife shield is movably located in shield guides formed by the walls of said penetrating head and said apex knife, and adapted to actuating between a retracted position, in which the apex knife is open, and an extended position, in which the apex knife is closed and protected by the apex knife shield. The dilating sloping surface, apex knife and apex knife shield protruding distally beyond the trocar cannula, are made without substantial gaps between them in the all position of the penetrating head, thereby preventing the tissue fibers from introducing, jamming or engagement between them during the all stages of penetrating a patient's body cavity wall. Specifically, the apex knife shield is disposed from that side of the apex knife, from which the sharpened cutting edge of the apex knife is disposed, thereby eliminating a gap between the sharpened cutting edge and the apex knife shield and preventing the tissue fibers of the patient's cavity wall from introducing, jamming or engagement between the apex knife shield and the apex knife. This heightens the reliability of the trocar operation. The indicated width, thickness and disposition of the apex knife shield provide the low resistance to the shield advance into the body tissue, enhancing the effectiveness of the safety means operation. The biasing spring is designed for permitting the movement of the apex knife shield towards the retracted position in response to a proximally directed force applied to the apex knife shield distal edge during penetrating the patient's body cavity wall, and for biasing the apex knife shield towards the extended position when the force applied to the apex knife shield distal edge is removed while the apex knife shield enters the patient's body cavity, however before the penetrating head has been fully inserted into the patient's body cavity. The safety means is made as a unified universal means with the width of the apex knife shield less than 5 mm suitable for 5 mm penetrating head maximal diameter and therefore applicable for the penetrating heads having the greater diameters. The performance of the safety means as a unified means along with the minimizing the safety means details due to their complete disposal at the penetrating head allow substantial reducing the trocar obturator cost and improving its manufacturing properties.

The trocar obturator is provided with a quickly acting connecting means allowing quick attachment/detachment of the safety means and apex knife to/from the penetrating head, as well as the penetrating head cover to/from the penetrating head base.

Moreover, there is an setting guide means designed for more convenient, simple and precise putting together the constituents of the penetrating head until they have been completely attached by the quickly acting connecting means. The quickly acting connecting means and the setting guide means provide the simplification and acceleration of the process of the reusable trocar disassembly and subsequent assembly, as well as the improvement of the convenience of this process performance. The apex knife shield, apex knife body and biasing spring are capable of being put together as a penetrating subassembly suitable for the replacement as a single unit. Using this single unit allows additional enhancing the convenience and speed of the trocar obturator assembly and disassembly. Specifically, the indicated single unit creates the favorable conditions for cost-effective application of the present trocar obturator in a partly reusable version, wherein only this single unit is a disposable component.

In version embodiment, the penetrating head is provided with lateral knives, which along with the apex knife body are made as a single member, and these lateral knives protrude from the dilating sloping surface through a slot in the penetrating head. The lateral knives facilitate the advance of the penetrating head into the body tissue. At the same time, their fabrication as the elements of the apex knife body maintains the all above advantages of the trocar obturator including its low cost, the easy replacement of its safety means and knives, and high reliability. There are also lateral shields designed for protecting the lateral knives. The lateral shields along with the apex knife shield are made as a single shield member. In the penetrating subassembly lying outside the trocar obturator, the shield member is in a preset position and the lateral knives are protected with the lateral shields. This allows safe attaching/detaching the penetrating subassembly to/from the penetrating head of the reusable trocar obturator. During attaching the penetrating subassembly to the trocar obturator, the shield member is transposed from the preset position to the extended position, wherein the lateral knives are not protected and ready for tissue cutting.

In version embodiment, the safety means also has the locking means for locking the apex knife shield after its returning in to the extended position, and subdivided into a locking mechanism including a distal abutment surface movable along with said apex knife shield and a proximal abutment surface immovable in axial direction relative to said obturator handle, a locking beam capable of moving between an extreme distal position wherein said apex knife shield is unlocked and extreme proximal position wherein it serves as a spacer between said abutment surfaces in said lock position eliminating the proximal axial displacement of said apex knife shield with respect to said shaft, and a control means designed for unlocking said apex knife shield by user.

In version embodiment, the safety means including abutment surfaces and control means are disposed in the limits of the penetrating head and along with the apex knife are constituents of a penetrating subassembly adapted for mounting on the shaft and there are a quickly acting connecting means allowing quick attachment/detachment of the penetrating subassembly to/from the shaft. This design has the control means made as a knob disposed on a proximal end of said locking beam, accessible for user after withdrawing the trocar obturator from the cannula, and allowing unlocking the apex knife shield by user's finger effort pressing the knob and displacing it distally.

In version embodiment, the penetrating subassembly along with the penetrating head form a single unit to be quick attached/detached to/from the shaft by quickly acting connecting means made as a collet connector.

In version embodiment, the control means has a control member disposed in the obturator handle and accessible for user without withdrawing trocar obturator from the cannula. In version embodiment, the trocar obturator has the penetrating head cover pivotally mounted on said penetrating head base and in this way inseparably connected to said shaft.

In version embodiment, the biasing spring is characterized by an axial force permitting to return said apex knife shield from the retracted position into the extended position after patient's skin penetration, but before a peritoneum penetration, therewith during moving through the patent's soft tissue including the peritoneum the apex knife shield serves as a blunt penetrating tip.

### BRIEF DESCRIPTION OF THE DRAWINGS

FIGS. 1 to 19 show the trocar obturator with detachable cutting means and safety means.

FIG. 1 shows the general side view of the trocar obturator with detachable apex knife body and apex knife shield.

FIG. 2 shows the close-up general side view of the trocar obturator penetrating head.

FIG. 3 shows the longitudinal section of the trocar obturator penetrating head.

FIG. 4 shows the side view of the penetrating head with the penetrating head base.

FIG. 5 shows the top view of the penetrating head with the penetrating head base.

FIG. 6 shows the longitudinal section of the penetrating head with the penetrating head base.

FIGS. 7, 8 show the cross sections of the penetrating head base.

FIG. 9 shows the side view of the penetrating head cover.

FIG. 10 shows the bottom view of the penetrating head cover.

FIG. 11 shows the longitudinal section of the penetrating head cover.

FIGS. 12, 13, 14 show the cross sections of the penetrating head cover.

FIG. 15 shows the longitudinal section of the clamping nut fixing the constituents of the penetrating head.

FIG. 16 shows the top view of the penetrating subassembly including the apex knife body, the apex knife shield and the biasing spring.

FIG. 17 shows the side view of the penetrating subassembly including the apex knife body, the apex knife shield and the biasing spring.

FIG. 18 shows the bottom view of the penetrating subassembly including the apex knife body, the apex knife shield and the biasing spring.

FIG. 19 shows the top view of the penetrating head with the clamping nut in unclamped state.

FIGS. 20 to 25 show the constituents of the trocar obturator with detachable cutting means and safety means designed for protecting a penetrating apex and lateral knives.

FIGS. 20 shows the side view of the penetrating subassembly including the apex knife body, the shield member and the biasing spring.

FIG. 21 shows the bottom view of the penetrating subassembly including the apex knife body, the shield member and the biasing spring.

FIG. 22 shows the top view of the penetrating subassembly including the apex knife body, the shield member and the biasing spring.

FIG. 23 shows the longitudinal section of the trocar obturator penetrating head with the clamping nut in unclamped position.

FIG. 24 shows the longitudinal section of the trocar obturator penetrating head in the early stage of screwing the clamping nut onto the penetrating head.

FIG. 25 shows the longitudinal section of the trocar obturator penetrating head after full screwing the clamping nut onto the penetrating head.

FIGS. 26 to 29 show the penetrating subassembly version including the locking means.

FIG. 26 shows the top view of the penetrating subassembly version.

FIG. 27 shows the cross-sectional view of the penetrating subassembly version.

FIG. 28 shows the side view of the penetrating subassembly version.

FIG. 29 shows the bottom view of the penetrating subassembly version.

FIGS. 30 to 35 show the penetrating head with the penetrating subassembly version including the locking means and the hinge connecting the penetrating head base and cover.

FIG. 30 shows the trocar obturator with the opened penetrating head cover and the removed penetrating subassembly.

FIG. 31 shows the penetrating head with the opened penetrating head cover and the installed penetrating subassembly.

FIG. 32 shows the penetrating head with unlocked apex knife shield in the extended position.

FIG. 33 shows the penetrating head with unlocked apex knife shield in the retracted position.

FIG. 34 shows the penetrating head with locked apex knife shield in the extended position.

FIG. 35 shows the penetrating head with the apex knife shield in the moment of its unlocking.

FIG. 36 shows the trocar obturator with the remote control of the locking means.

FIG. 37 shows the trocar obturator with the detachable penetrating head having the releasable collet connection with the shaft.

### DETAILED DESCRIPTION OF THE INVENTION

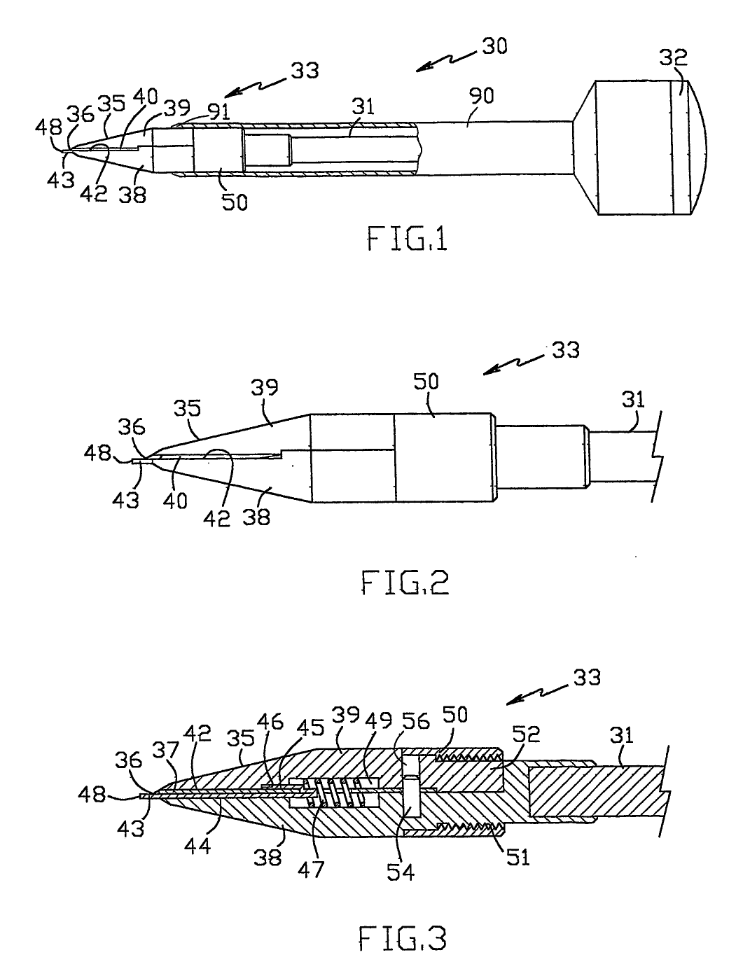

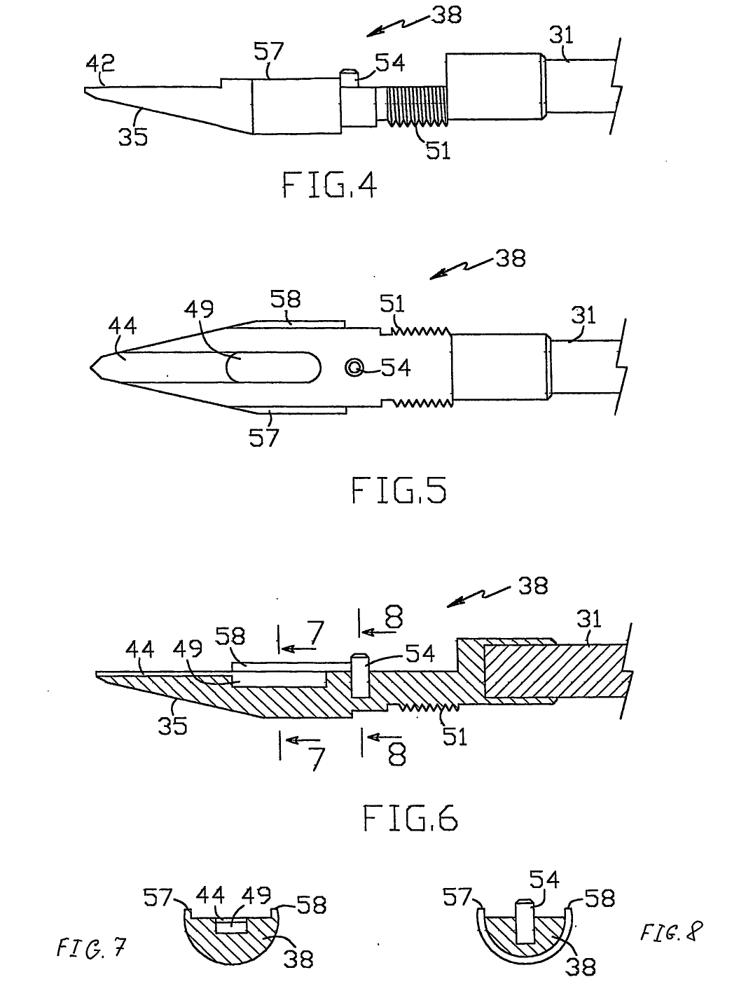

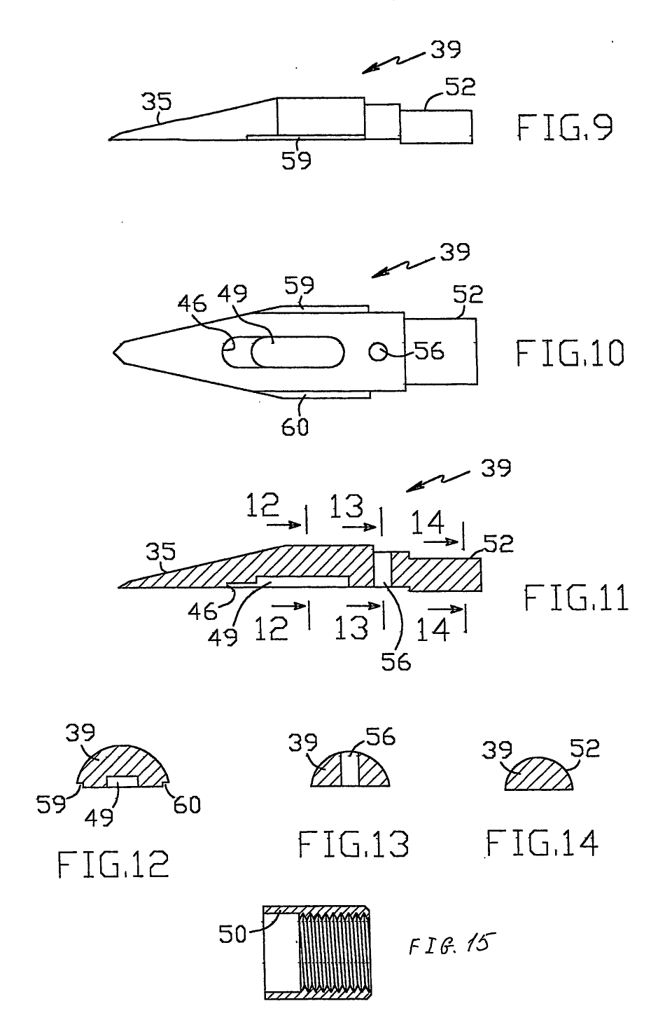

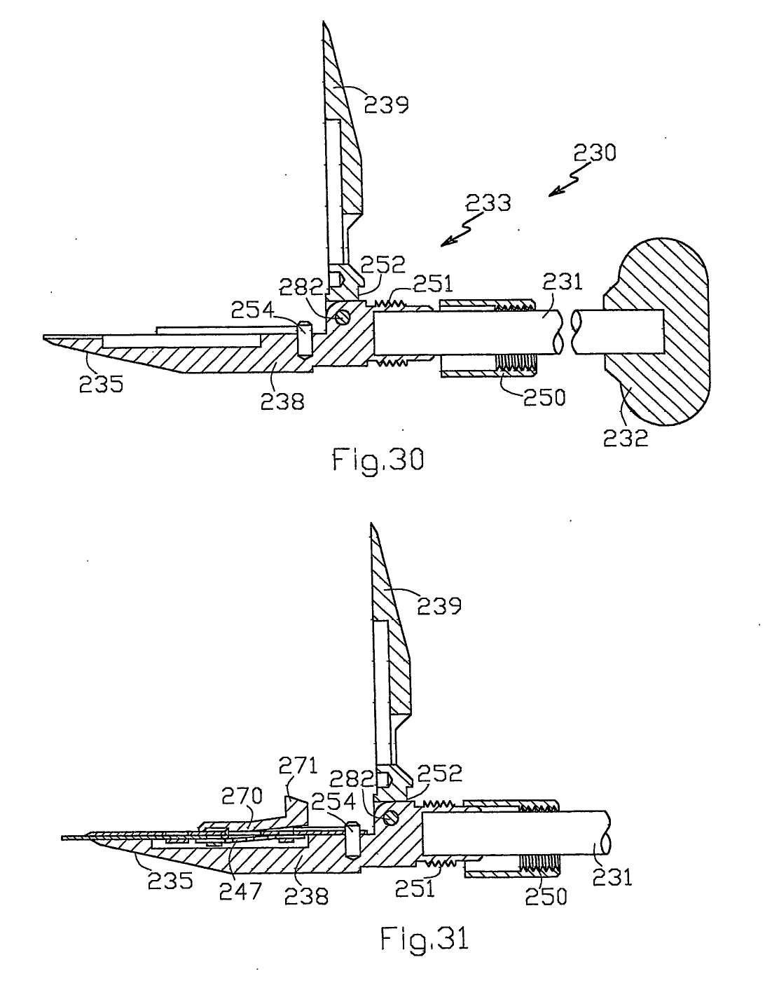

The description of the present invention is offered with the references made to the attached drawings in FIGS. 1 to 37.

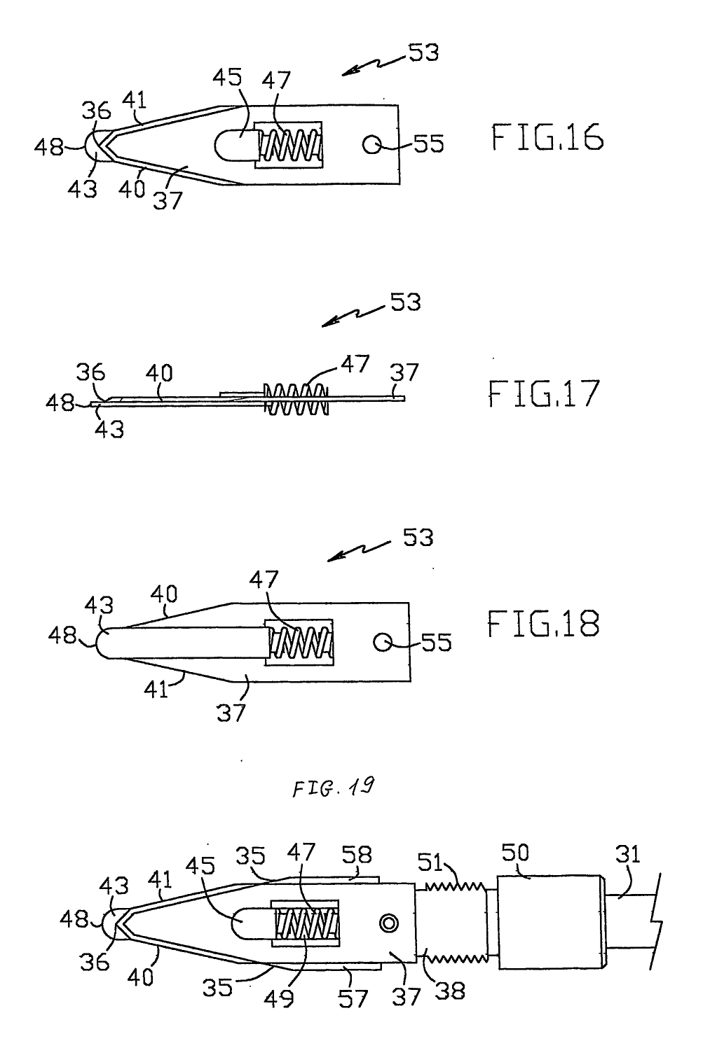

Trocar obturator 30, shown in FIG. 1, adapted to removable inserting into a cannula (not shown) and subsequent forming a passageway in a patient's body cavity wall, includes: shaft 31, obturator handle 32 disposed on the proximal end of shaft 31, and penetrating head 33 disposed on the distal end of shaft 31 and partly protruding distally beyond the distal end of the cannula. Penetrating head 33 (FIGS. 2, 3) has: dilating sloping surface 35 of conical form; apex knife with sharpened cutting edge 36 adapted to carrying out an incision In the body cavity wall, located on apex knife body 37 made as a flat plate and detachably housed in penetrating head 33, therewith apex knife 36 and dilating sloping surface 35 are immovable in the shaft axial direction relative to shaft 31 during the trocar operation; a safety means for protecting at least apex knife 36. Penetrating head 33 includes penetrating head base 38 (FIGS. 4 to 8) inseparably connected to shaft 31 and penetrating head cover 39 (FIGS. 9 to 14) quickly detachable from penetrating head base 38. Trocar obturator 30 is provided with two lateral knives 40, 41 protruding from dilating sloping surface 35. Lateral knives 40, 41 and apex knife body 37 are made as a single detail and the lateral knives protrude from sloping surface 35 through slot 42 formed between penetrating head base 38 and penetrating head cover 39. Lateral knives 40, 41 (FIGS. 16-19) facilitate the advance of penetrating head 33 into the body tissue. At the same time, their fabrication as the elements of apex knife body 37 allows maintaining the all advantages of the trocar obturator including its low cost, the easy replacement of its safety means and knives, and high reliability.

The safety means is completely disposed at penetrating head 33 and has flat apex knife shield 43 designed for the protection of apex knife 36 and adapted to actuating between a retracted position (not shown), in which apex knife 36 is open, and an extended position (FIGS. 1 to 3), in which apex knife 36 is closed and protected by apex knife shield 43. Shield 43 is movably located in shield guides formed by the walls of apex knife body 36 and longitudinal guide groove 44 made in a distal portion of penetrating head base 38 on its surface faced penetrating head cover 39. Therewith, there are no substantial gaps between apex knife shield 43 and the walls of guide groove 44. Apex knife shield 43 is disposed on that side of apex knife 36, on which the sharpened cutting edge of apex knife 36 is disposed, thereby eliminating a gap between the sharpened cutting edge of apex knife 36 and apex knife shield 43 and preventing the tissue fibers of the patients cavity wall from introducing, jamming or engagement between apex knife shield 43 and apex knife body 37. As a whole, the all elements of penetrating head 33 protruding distally beyond the trocar cannula, including dilating sloping surface 35, apex knife 36 and apex knife shield 43, are made without substantial gaps between them in the all position, thereby preventing the tissue fibers from introducing, jamming or engagement between them during the all stages of penetrating a patients body cavity wall. This heightens the reliability of the trocar operation. Apex knife shield 43 has the width substantially equal to the width of apex knife 36 and less than maximal diameter of penetrating head 33, and moreover, the apex knife shield width more than twice exceeds its thickness. The performance of apex knife shield 43 as a flat plate having a small cross-sectional area due to its indicated width and thickness, as well as its indicated disposition from one side of apex knife 36 provide the low resistance to the apex knife shield advance into the body tissue, enhancing the effectiveness of the safety means operation. Projection 45 of apex knife shield 43 is adapted to abutting against ledge 46 in penetrating head cover 39 eliminating the displacement of apex knife shield 43 distally of its extended position. The safety means also comprises biasing spring 47 for permitting the movement of apex knife shield 43 towards the retracted position in response to a proximally directed force applied to shield distal edge 48 during penetrating the patient's body cavity wall, and for biasing apex knife shield 43 towards the extended position, when the force applied to shield distal edge 48 is removed while apex knife shield 43 enters the patient's body cavity, however before dilating sloping surface 35 has been fully inserted into the patient's body cavity. Biasing spring 47 is located between penetrating head parts 38, 39 in longitudinal recess 49 made partly in penetrating head base 38 and partly in penetrating head cover 39.

In version embodiment, the safety means is made as an unified universal means with the width of apex knife shield 43 less than 5 mm suitable for 5 mm maximal diameter of penetrating head 33 and therefore applicable for the penetrating heads of greater diameters. The performance of the safety means as an unified means along with the minimizing the safety means details 43, 47 due to their complete disposal at penetrating head 33 allow substantial reducing the trocar obturator cost and improving its manufacturing properties. In another version embodiment (not shown), there is an additional apex knife shield located on another side of the apex knife, therewith both shields have a common base and are biased by a common biasing spring.

In another version embodiment (not shown), the apex knife shield is tubular.

In another version embodiment (not shown), the apex knife shield and the biasing spring are made as a single detail.

In another version embodiment (not shown), the apex knife, apex knife shield and biasing spring are made as a single detail.

Trocar obturator 30 is provided with a quickly acting connecting means allowing quick attachment/detachment of the safety means including apex knife shield 43 and biasing spring 47 as well as apex knife body 37 to/from penetrating head 33. The quickly acting connecting means includes a thread clamping nut 50, a connecting thread 51 located on a proximal portion of penetrating head base 38, and proximal shank 52 of penetrating head cover 39 adapted to clamping by thread clamping nut 50. The latter also relates to the mounting means for holding the penetrating head constituents in the closed position. Apex knife shield 43, apex knife body 37 and biasing spring 47 are capable of being put together as penetrating subassembly 53 (FIGS. 16 to 19) suitable for the replacement as a single unit.

Penetrating head 33 includes a setting guide means designed for more convenient, simple and precise putting together the constituents of the penetrating head until they have been completely secured by the mounting means. The setting guide means include guide pin 54 located on penetrating head base 38 and designed for entering guide opening 55 in apex knife body 37 and guide opening 56 in penetrating head cover 39, as well as lateral protrusions 57, 58 on penetrating head base 38 designed for the lateral guidance of penetrating subassembly 53 and for entering notches 60, 59 on penetrating head cover 39.

The quickly acting connecting means and the setting guide means facilitate the trocar obturator assembling, lowering its manufacturing cost. Moreover, they provide the simplification and acceleration of the reusable trocar disassembly and subsequent assembly, as well as the improvement of the operation convenience. Using single unit 53 allows additional enhancing the convenience and speed of the trocar obturator assembly and disassembly. Specifically, single unit 53 creates the favorable conditions for cost-effective application of trocar obturator 30 in a partly reusable version, wherein only single unit 53 is a disposable component.

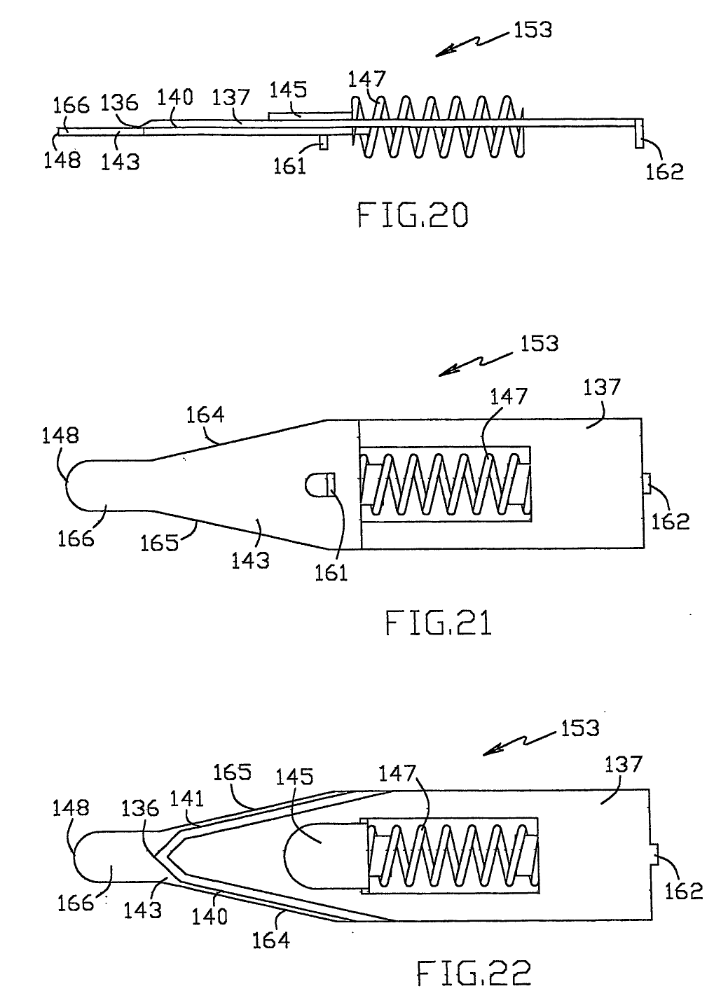

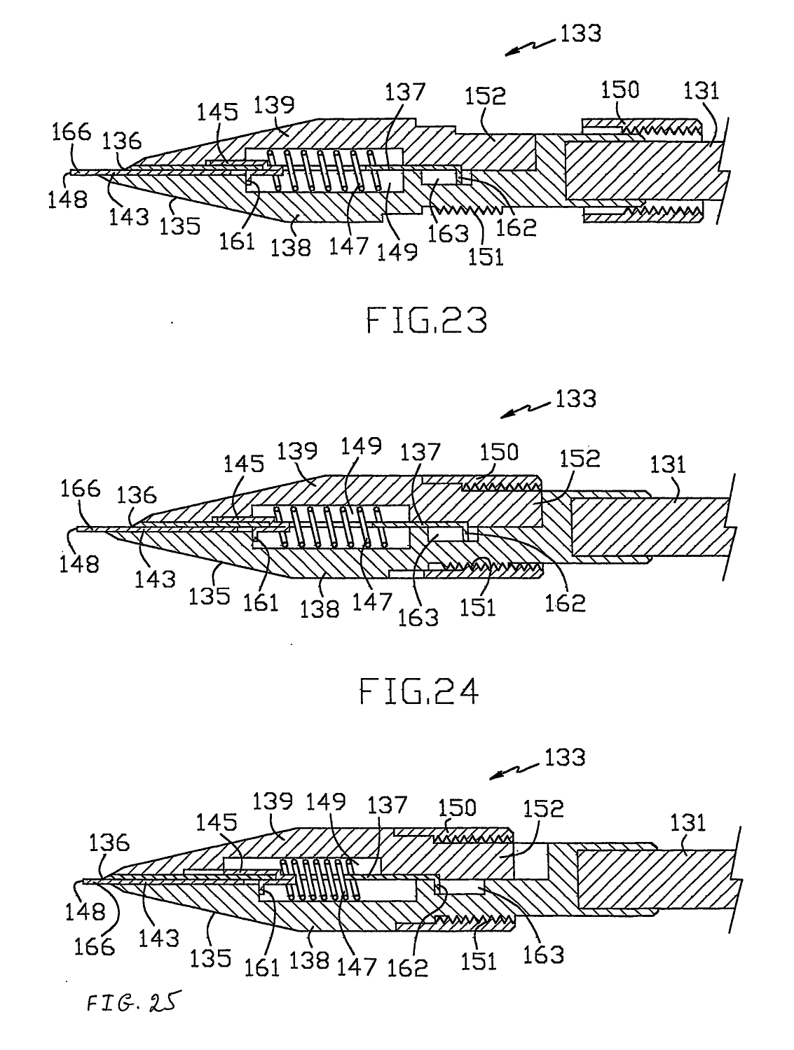

In version embodiment, shown in FIGS. 20 to 25, penetrating head 133 includes penetrating subassembly 153 comprising apex knife body 137 with apex knife 136 and two dilating lateral knives 140, 141, as well as shield member 143 with lateral shields 164,165, whose outer edges are equidistant to lateral knives 140, 141, respectively. There is also biasing spring 147, mounted between apex knife body 137 and shield member 143. In penetrating subassembly 153 lying outside penetrating head 133 (FIGS. 20 to 22), shield member 143 is in the preset position, wherein it protects apex knife 136 by shield distal protrusion 166, as well as lateral knives 140, 141 by lateral shields 164, 165, respectively (FIG. 22). During mounting penetrating subassembly 153 into penetrating head 133 (FIGS. 23 to 25), apex knife body 137 along with penetrating head cover 139 is displaced distally relative to shield member 143 by clamping nut 150. In doing so, clamping nut 150 transmits distally directed axial movement to apex knife body 137 through penetrating head cover 139 and transmitting element 162 of apex knife body 137, compressing biasing spring 147, while shield member 143 is immovable due to restricting element 161 of shield 143. Restricting element 161 and transmitting element 162 relate to the installing means allowing to set shield member 143 into its extended position relative to apex knife body 137 as a result of screwing clamping nut 150 on penetrating head base 138. In the shield member extended position (FIG. 25), lateral knives 140, 141 are not protected with shield member 143, while penetrating apex 136 remains protected with distal protrusion 166 of shield member 143. Henceforward, in the assembled state of penetrating head 133, lateral knives 140, 141 are constantly not protected, and shield distal protrusion 166 operates identically to shield 43 of previous penetrating head 33, protecting or releasing apex knife 136. After surgery performance, disassembly of penetrating head 133 and withdrawal of penetrating subassembly 153, shield member 143 returns in its preset position, shown in FIG. 22, providing the personnel protection from any contact with sharp edges of apex knife body 137. Penetrating head 133 has a setting guide means designed for more convenient, simple and precise putting together the penetrating head parts 138, 139 and penetrating subassembly 153 until they have been completely attached by the quickly acting connecting means. The setting guide means include recess 163 in penetrating head base 138, wherein transmitting element 162 is movably housed, as well as the lateral protrusions on penetrating head base 138 (not shown) identical to lateral protrusions 57, 58 (FIGS. 5, 7, 19) for lateral guidance of penetrating subassembly 153. The version embodiments, shown in FIGS. 1 to 19 and FIGS. 20 to 25, comprise many identical details having the same two last numerals in their designations. The description of these details, made for FIGS. 1 to 19, can be also used for the details of FIGS. 20 to 25, if they have not their own description.

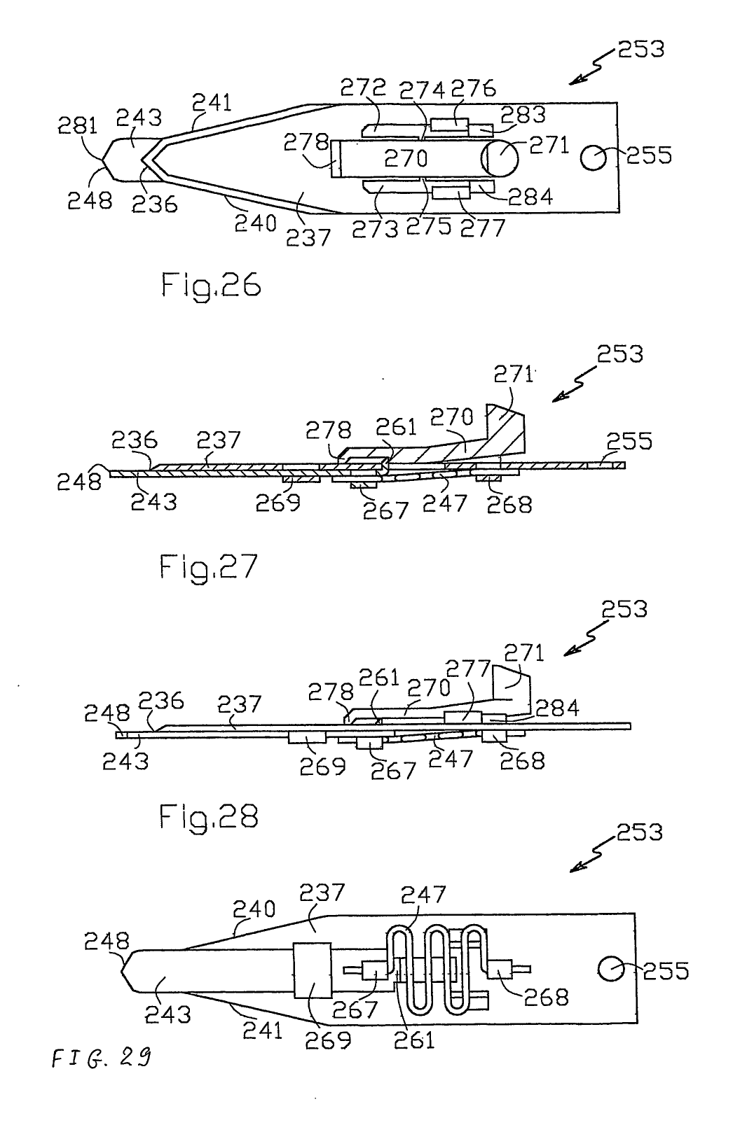

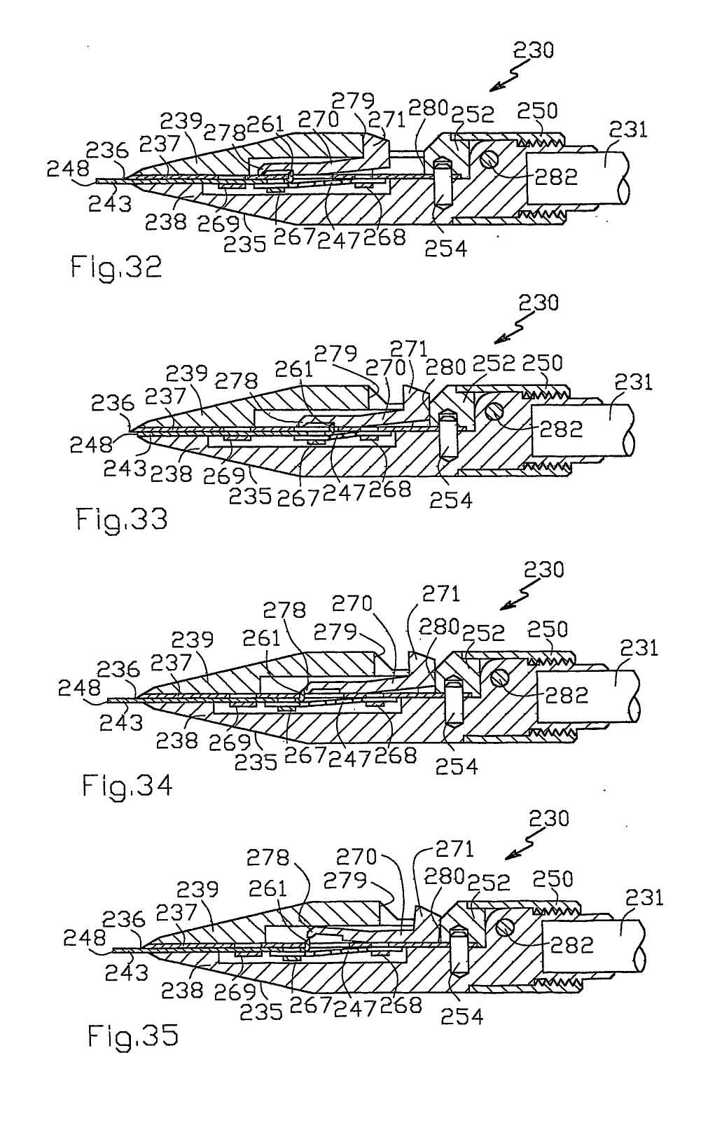

In version embodiment, shown in FIGS. 26 to 35, penetrating subassembly 253 includes apex knife 236 wits its apex knife body 237, apex knife shield 243 having shield distal edge 248 and movably housed in guide 269 made as an integral part of apex knife body 237, as well as biasing spring 247 connected with apex knife shield 243 by keeper 267 and with apex knife body 237 by keeper 268. Penetrating subassembly 253 also comprises a locking means designed for locking apex knife shield 243 in its extended position at least after piercing a patients skin by apex knife 236. The locking means comprises a locking mechanism, as well as a control means designed for unlocking apex knife shield 243 by user at his own choosing. The locking mechanism includes locking beam 270 capable of moving in the axial direction with respect to shaft 231 between an extreme proximal position (FIG. 34) wherein locking beam 270 is fixed by a fixation means and locks apex knife shield 243 in its extended position and an extreme distal position (FIG. 32) wherein apex knife shield 243 is unlocked. To provide the axially directed movement, locking beam 270 has a carriage in the form of two carriage members 272, 273 movably located in carriage guides 276, 277 made as integral parts of apex knife body 237. The contacting friction surfaces of carriage members 272, 273 and carriage guides 276, 277 serve as the fixation means providing the fixation of locking beam 270 in its extreme proximal position at the expense of friction force. Carriage members 272, 273 are provided with restricting protrusions 283, 284 restricting the distal axial movement of locking beam 270 relative to apex knife body 237 and serving as additional components of the above setting guide means facilitating the emplacement of penetrating subassembly 253. Carriage members 272, 273 are connected with locking beam 270 by elastic links 274, 275 which allow turning locking beam 270 around the axis transverse relative to the axis of shaft 231 and passing through elastic links 274, 275. Therewith, the locking beam turning (FIG. 35) is implemented by user through the control means to unlock apex knife shield 243. Thus the locking mechanism is disposed at a distal portion of shaft 231 in the limits of penetrating head 233 and is a constituent of penetrating subassembly 253 capable of emplacing into penetrating head 233 and removing therefrom along with penetrating subassembly 253 as its integral part.

Locking beam 270 is provided with locking projection 278 disposed at a locking beam distal end and interacting with abutment member 261 located on the proximal end of apex knife shield 243. Therewith the distal surface of abutment member 261 and the proximal surface of locking projection 278 are sloping thereby allowing the unhampered transposition of apex knife shield 243 from the retracted position to the extended position elevating locking projection 278 (FIG. 35) due to turning locking beam 270 around the transverse axis. Moreover, the proximal surface of abutment member 261 and the distal surface of locking projection 261 are vertical, thereby hampering the transposition of apex knife shield 243 from the extended position to the retracted position and locking apex knife shield 243 in the extended position. In this lock position, the proximal surface of abutment member 261 serves as a distal abutment surface of locking beam operating as a spacer between apex knife shield 243 and the details immovable relative to the obturator handle.

The control means of the locking means is made as knob 271 disposed on the proximal end of locking beam 270, accessible for user after withdrawing said trocar obturator from cannula, and allowing unlocking apex knife shield 243 by user's finger effort pressing knob 271 and displacing it distally. Knob 271 is made as an integral part of locking beam 270 and along with it is the integral constituent of penetrating subassembly 253. Knob 271 is disposed within a recess in penetrating head cover 239, distal 279 and proximal 280 walls of the recess serve as the limiters of the axial transposition of knob 271 thereby determining the extreme distal and proximal positions of locking beam 270. Proximal wall 280 serves as a proximal abutment surface of locking beam, when it operates as the spacer In the lock position.

In version embodiment, biasing spring 247 Is characterized by an axial force permitting the displacement of apex knife shield 243 into the retracted position in response to a proximally directed force applied to apex shield distal edge 248 during penetrating a patient's skin, and biasing apex knife shield 243 into the extended position with its locking in this position when the force applied to apex shield distal edge 248 is decreased as a result of moving apex knife shield 243 in a patients soft tissue. Therewith, during moving in the patient's soft tissue, locked apex knife shield 243 serves as a blunt penetrating tip of penetrating head 253 thereby substantially heightening the operation safety. The distal edge of apex knife shield 243 has a configuration facilitating its passing through a patient's tissues but sufficiently safe for the patients organs located in its body cavity. For this purpose, distal edge 248 is pointed, therewith its configuration is formed with the face surfaces converging to line 281 at obtuse angle and at distal face 248 is made non-cutting.

The method of this trocar operation comprises: forming an initial incision in the patient's skin with the apex knife236, therewith in doing so the skin displaces apex knife shield 243 in said retracted position; continuation of forming the passageway in the patient's soft tissue, while apex knife shield 243 is displaced into the extended position by biasing spring 247 due to that the force of the biasing spring exceeds the resistance force of the patient's soft tissue; and during moving through the patient's soft tissue apex knife shield 243 begins to operate as a blunt penetrating tip so that a peritoneum is penetrated by It. Therewith, apex knife 243 has locking means locking the apex knife shield in the extended position before the apex knife shield entering the body cavity, thereby enhancing the reliability of operating apex knife shield 243 as the penetrating blunt tip. As a result, the injury of patient's internal organs by apex knife 236 is completely eliminated.

In another version, biasing spring 247 is characterized with an axial force permitting the displacement of apex knife shield 243 into the retracted position in response to a proximally directed force applied to apex shield distal edge 248 during penetrating the patient's skin and soft tissue, and biasing apex knife shield 243 into the extended position when the force applied to apex shield distal edge 248 is removed while apex knife shield 243 enters a patient's body cavity, however before dilating sloping surface 235 has been inserted into the patient's body cavity.

As applied to trocar obturator version 230, the setting guide means include hinge 282 connecting penetrating head cover 239 and penetrating head base 238 allowing opening penetrating head parts 238, 239 for removing contaminated penetrating subassembly 253 and closing the penetrating head pats after emplacing the cleaned penetrating subassembly without full disengaging penetrating head parts 238, 239. This provides improving the operation convenience and reducing the time needed for assembling penetrating head 233 after its cleaning and disinfecting.

Penetrating head versions 33, 133 and 233 have many identical details with the same two last numerals in their designations. The description of these details made for the versions 33, 133 is applicable also for version 233.

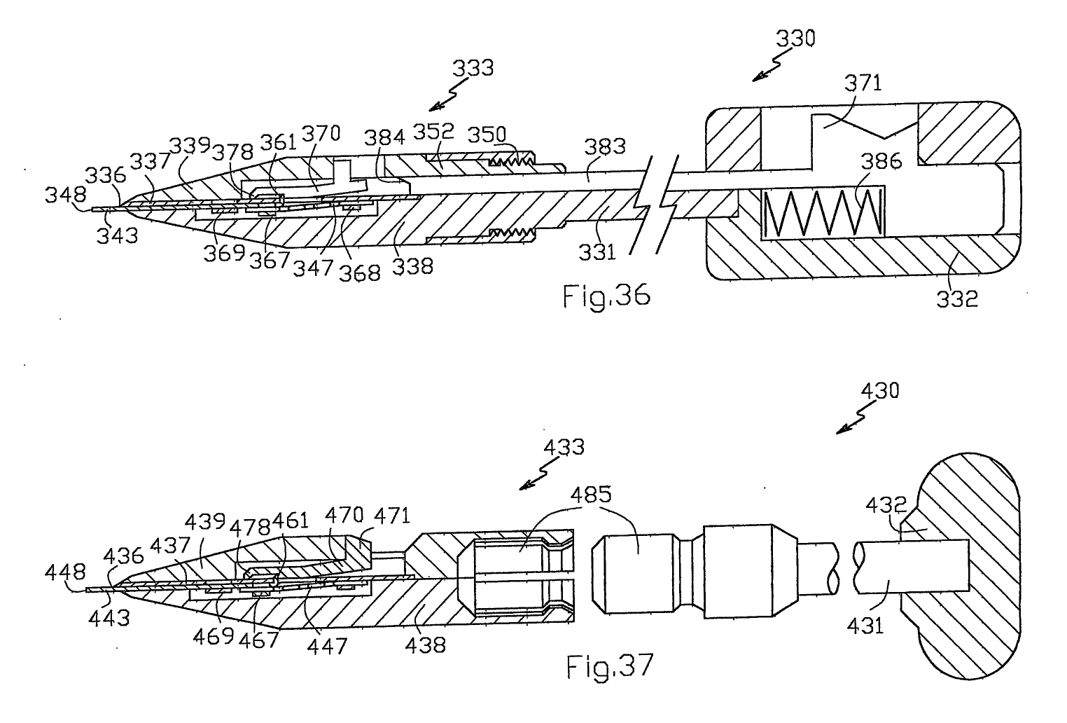

Version embodiment of trocar obturator 330, shown in FIG. 36, comprises the penetrating subassembly identical to one of version 230 with the exception of the control means, which is made as a remote means including remote knob 371 disposed in obturator handle 332 and accessible for user, control spring 386 and transmitting bar 383. The latter has proximal end connected with remote knob 371 and distal face 384 adapted to the contact interaction with the proximal end of locking beam 370 and configured to provide first the locking beam turning around the transverse axis and then transposing locking beam 370 from the extreme proximal position to the extreme distal position during transposing transmitting bar 383 distally by user effort applied to control knob 371 and directed distally to unlock apex knife shield 343. Version 330 is disposal and includes penetrating head base 338 and penetrating head cover 339 inseparably connected to one another after installing the penetrating subassembly. Therewith, the control means of such type can be used also in the reposable version wherein the penetrating subassembly is disposable and the obturator handle is reusable.

The trocar obturator version 430, shown in FIG. 37, has a quickly acting collet connection 485 allowing quick attachment/detachment of penetrating head 433 as a whole to/from shaft 431. Penetrating head 433 includes penetrating head base 438 and penetrating head cover 439 inseparably connected to one another after emplacement the penetrating subassembly, which is identical to above penetrating subassembly 253. Penetrating head 433 is designed for the application as a disposal trocar component, while shaft 431 can be reusable. Therewith, the same one shaft 431 can be applied with the penetrating heads of various maximal diameters.

Obviously, numerous other variations and modifications can be made without departing from the spirit of the present invention. Therefore, it should be clearly understood that the forms of the present invention described above and shown in the figures of the accompanying drawings are illustrative only and are not intended to limit the scope of the present invention.

1-45. (canceled)

46. A safety trocar obturator adapted to removable inserting into a cannula and subsequent forming a passageway in a patient's body cavity wall, including:
a shaft having:
a distal end and
a proximal end;
an obturator handle disposed on said shaft proximal end;
a penetrating head disposed on said shaft distal end and including:
a dilating sloping surface protruding distally beyond the distal end of said cannula;
a penetrating subassembly adapted for mounting on said penetrating head and including:
an apex knife with sharpened cutting edge;
a safety means for said apex knife protection including:
an apex knife shield having:
a retracted position, in which said apex knife is open, and an extended position, in which said apex knife is protected;
a biasing spring urging said shield from said retracted position to said extended position;
said shield and said apex knife cutting edge protruding distally beyond said dilating sloping surface.

47. The trocar obturator of claim 46, has a quickly acting connecting means allowing quick attachment/detachment of said penetrating subassembly to/from said shaft distal end.

48. The trocar obturator of claim 47, wherein said apex knife shield is tubular and designed to surround said apex knife.

49. The trocar obturator of claim 47, wherein said apex knife is flat, said apex knife shield is flat and adjoins to said apex knife from the side of said sharpened cutting edge.

50. The trocar obturator of claim 47, wherein said penetrating head has a penetrating head base presenting a constituent of said quickly acting connecting means and inseparably connected to said shaft, and said penetrating subassembly is adapted for mounting on said shaft through said penetrating head base, therewith said penetrating head base and said penetrating subassembly have the geometry of contacting portions, which facilitates their matching and quick assembling.

51. The trocar obturator of claim 50, wherein said quickly acting connecting means includes a penetrating head cover, and said penetrating subassembly is mounted between said head cover and said penetrating head base.

52. The trocar obturator of claim 51, wherein said penetrating head cover is pivotally mounted on said penetrating head base.

53. The trocar obturator of claim 47, wherein said penetrating head is connected to said shaft by said quickly acting connecting means allowing quick attachment/detachment of said penetrating subassembly to/from said shaft along with said penetrating head as a whole.

54. The trocar obturator of claim 53, wherein said quickly acting connecting means is made as a collet connector.

55. The trocar obturator of claim 47, wherein there is at least one lateral knife protruding from said dilating sloping surface.

56. The trocar obturator of claim 46, wherein said apex knife shield and biasing spring are made as a single detail.

57. The trocar obturator of claim 56, wherein said apex knife, apex knife shield and biasing spring are made as a single detail.

58. The trocar obturator of claim 46, wherein said apex knife is pointed to reduce force applied on said body cavity wall during its penetration.

59. The trocar obturator of claim 46, wherein said biasing spring is characterized by an axial force permitting to return said apex knife shield from said retracted position into said extended position after patient's skin penetration, but before a peritoneum penetration, therewith during moving through the patient's soft tissue including the peritoneum said apex knife shield serves as a blunt penetrating tip.

60. The trocar obturator of claims 59, wherein there is a locking means, which locks said apex knife shield after returning said apex knife shield into said extended position thereby enhancing its operation reliability as a blunt penetrating tip.

61. The trocar obturator of claims 59, wherein said apex knife shield has a pointed distal edge to ease its passing through a cavity wall tissues, but said pointed distal edge is formed with surfaces disposed at obtuse angle to one another to provide its safety for patient's internal organs.

62. The trocar obturator of claim 46, including:
a locking means for locking said apex knife shield in its extended position, having:
a lock position,
an unlock position;
a locking mechanism having a distal abutment surface and proximal abutment surface disposed in the limits of said penetrating head;
said distal abutment surface movable along with said apex knife shield,
said proximal abutment surface immovable in axial direction relative to said obturator shaft, therewith said lock position of locking means is provided when said distal abutment surface is butted up against said proximal abutment surface.

63. The trocar obturator of claim 62, wherein said abutment surfaces have
a mediate contact with one another by means of a spacer placed between them and movable between a distal position wherein said apex knife shield is unlocked in said extended position and an extreme proximal position wherein said apex knife shield is locked in said extended position,
and said spacer is made as a locking beam having:
a beam distal end and
a beam proximal end, therewith in said locking position said beam distal end interacts with said distal abutment surface and through it with said apex knife shield and said beam proximal end interacts with said proximal abutment surface locking said apex knife shield in said extended position.

64. The trocar obturator of claim 63, wherein said locking means includes a control means to unlock said apex knife shield and to return said locking means from said lock position to said unlock position, and said apex knife shield unlocking is implemented by disengagement of said locking beam from said abutment surfaces.

65. The trocar obturator of claim 64, wherein said control means is disposed in the limits of said penetrating head and made as a knob located on a proximal end of said locking beam and accessible for user after withdrawing said trocar obturator from said cannula.

66. The trocar obturator of claim 65, wherein said penetrating subassembly includes said locking mechanism and there is a quickly acting connecting means allowing quick attachment/detachment of said penetrating subassembly to/from said penetrating head.

67. The trocar obturator of claim 64, wherein said control means is made as a remote means including:
a remote knob disposed on said obturator handle,
a control spring and
a transmitting bar having:
a proximal end connected to said remote knob and
a distal end adapted to the contact interaction with said locking beam proximal end.

68. A method of forming a passageway in a patient's body cavity wall by a trocar, including a safety trocar obturator removably inserted into a cannula and having:
a penetrating head having:
a sloping surface protruding distally beyond a distal end of said cannula;
an apex knife with sharpened cutting edge;
a safety means for said apex knife protection including:
an apex knife shield having:
a retracted position, in which said apex knife is open,
and an extended position, in which said apex knife is protected;
a biasing spring urging said shield from said retracted position to said extended position;
said method comprises:
pressing onto patient's skin by said trocar directed substantially perpendicularly to patient's skin leading to displacing said apex knife shield in said retracted position and baring said apex knife,
penetrating the patient's skin with said apex knife,
penetrating the remaining patient's soft tissues layers including a peritoneum by said penetrating head, which is blunt due to that immediately after skin penetrating said apex knife shield is returned in said extended position by said biasing spring whose force exceeds the resistance force of the patient's soft tissue, and as a result, during moving through the patient's soft tissue said apex knife shield operates as a penetrating blunt tip.
